(19) United States
(12) Patent Application Publication
(10) Pub. No.: US 2020/0289459 A1
(43) Pub. Date: Sep. 17, 2020

(54) LIQUID DOSAGE FORMS, METHODS OF MAKING AND USE

(71) Applicant: CANOPY GROWTH CORPORATION, Smiths Falls (CA)

(72) Inventors: Ben GEILING, Smiths Falls, Ontario (CA); Tom SHIPLEY, Smiths Falls, Ontario (CA)

(21) Appl. No.: 16/651,272

(22) PCT Filed: Nov. 30, 2018
(86) PCT No.: PCT/CA2018/051533

§ 371 Date: Mar. 26, 2020

Related U.S. Application Data
(60) Provisional application No. 62/592,993, filed on Nov. 30, 2017.
(60) Provisional application No. 62/611,851, filed on Dec. 29, 2017.
(60) Provisional application No. 62/618,790, filed on Jan. 18, 2018.
(60) Provisional application No. 62/632,272, filed on Feb. 19, 2018.
(60) Provisional application No. 62/632,279, filed on Feb. 19, 2018.
(60) Provisional application No. 62/632,286, filed on Feb. 19, 2018.
(60) Provisional application No. 62/632,289, filed on Feb. 19, 2018.
(60) Provisional application No. 62/632,295, filed on Feb. 19, 2018.
(60) Provisional application No. 62/632,299, filed on Feb. 19, 2018.
(60) Provisional application No. 62/632,309, filed on Feb. 19, 2018.

(51) Int. Cl. A61K 31/352; A61K 31/05; A61K 9/107; A61K 9/14

(52) U.S. Cl. CPC A61K 31/352; A61K 9/14; A61K 9/1075; A61K 31/05

(57) ABSTRACT

Cannabis and cannabis derived formulations, methods of making and methods of using the formulation in oral dosage forms, preferably in beverage forms, are disclosed herein.

### CROSS-REFERENCE TO RELATED APPLICATIONS

This application claims priority from U.S. Provisional Application Ser. No. 62/592,993, filed Nov. 30, 2017, U.S. Provisional Application Ser. No. 62/611,851, filed Dec. 29, 2017, U.S. Provisional Application Ser. No. 62/618,790, filed Jan. 18, 2018, U.S. Provisional Application Ser. No. 62/632,272, filed Feb. 19, 2018, U.S. Provisional Application Ser. No. 62/632,279, filed Feb. 19, 2018, U.S. Provisional Application Ser. No. 62/632,286, filed Feb. 19, 2018, U.S. Provisional Application Ser. No. 62/632,289, filed Feb. 19, 2018, U.S. Provisional Application Ser. No. 62/632,295, filed Feb. 19, 2018, U.S. Provisional Application Ser. No. 62/632,299, filed Feb. 19, 2018, and U.S. Provisional Application Ser. No. 62/632,309, filed Feb. 19, 2018, the disclosures of which are hereby incorporated by reference in their entireties.

### BACKGROUND

The present disclosure is directed to compositions, methods of making and methods of use of cannabis and cannabis derived compositions in oral dosage forms, such as for use in foods and beverages. The dosage forms of the disclosure provide compositions, methods of making and methods of use of cannabis and cannabis derived compositions which alleviate many if not all the unwanted effects presented by smoking or inhaling vaporized substances from cannabis in whole plant or processed plant forms.

Smoking is not typically acceptable to non-smokers, it can be aesthetically unpleasant, and can involve health risks such as irritation to at least the mouth, esophagus and lungs. Cigarette smoking has been linked to devastating health risks thought to result from the formation of harmful combustion products. In some jurisdictions legislation exists, which prohibits smoking in various locations and cannabis smoking itself is the target of regulation due to so called “second hand smoke” risks as well as what is said to be unpleasant smells for some people. Methods for consuming cannabis and cannabis derived compositions which do not involve smoking or other vaporous means of ingestion are advantageous because such methods do not involve these and other unwanted effects.

Oral consumption comprises a significant percentage of total cannabis use in federally legal jurisdictions as well as on a state, province, or the like, basis globally. Many orally consumable products, however, contain unhealthy amounts of substances other than cannabis or cannabis derived compositions. Such ingredients include various sugars, caffeine and a variety of non-sugar stimulants, ethanol, and plant based substances thought to be nutritional supplements, but which have not been the subject of extensive safety testing in complex formulations including cannabis and cannabis derived compositions. Further, known oral products use expensive gums, which have unpredictable supply.

Based on the foregoing, it would be advantageous for a convenient formulation of cannabis and cannabis-derived compounds (e.g., cannabinoids) for use in beverages and food. More particularly, it would be beneficial to provide a formulation of cannabinoids for use in liquid, powder and solid forms. The formulation would advantageously have little or no taste and odor. In some aspects, in addition to the cannabinoids, the formulation could include other cannabis-derived molecules (e.g., terpenes) and nutritional supplements (e.g., vitamins) in a single convenient dosage form.

### BRIEF DESCRIPTION OF THE DISCLOSURE

The present disclosure is directed to formulations including cannabis and cannabis-derived compounds for use in beverages and foods, and to methods of preparing the formulations. Most suitably, the formulations are physically and chemically stable and have minimal flavor. Further, the formulations include favorable pharmacokinetics, for example, rapid onset, shorter duration, and minimal food effect as described more fully herein.

In some embodiments, the cannabinoid formulations are in liquid form. In other embodiments, the cannabinoid formulations are in solid form, such as in powder form configured to be added to foods and liquid beverages.

More particularly, in one aspect, the present disclosure is directed to a liquid formulation comprising at least one cannabis or cannabis derived compound and a biocompatible liquid.

In another aspect, the present disclosure is directed to an emulsion comprising the liquid formulation of at least one cannabis or cannabis derived compound, a carrier oil, and an aqueous solution.

In yet another aspect, the present disclosure is directed to a method of preparing the liquid formulation, the method comprising: mixing at least one cannabis or cannabis derived compound and a biocompatible liquid. In some embodiments, the methods further include drying the formulations to form a dry powder formulation for addition to foods and beverages.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the suitable methods and materials are described below.

As used in this application, including the appended claims, the singular forms “a,” “an,” and “the” include plural references, unless the content clearly dictates otherwise, and are used interchangeably with “at least one” and “one or more.”

The president disclosure is generally directed to cannabis and cannabis-derived compound containing formulations in oral dosage forms and to methods of preparing the formulations. The formations are suitably in a nontoxic consumable liquid or powder form. Suitably, the formulations provide stability, have minimal flavor and odor, and are natural in origin.

The formulations include one or more or cannabis and cannabis-derived compounds. Cannabis has been used in beverage preparations for millennia. Most of historical cannabis beverages were prepared by boiling or grinding cannabis leaves, combining with water, milk, alcohol, or another biocompatible matrix and, optionally, mixing with herbal or other plant-based compositions to form the final consumable. The present disclosure provides improvements over historical cannabis-containing beverages in at least two ways. First, the formulations of the present disclosure are prepared using a great variety of methods not available historically. Because many new methods of preparing cannabis and cannabis derived compositions are used, the chemical and physical makeup of the compositions are different and developed for specific use in oral dosage forms. Secondly, the precise makeup of the oral dosage forms uses additives and modifiers which were not available historically.

Individually and separately these improvements produce dosage forms, and, at times, the combinations of ingredients can provide synergistic beneficial effects on preparation, storage, distribution or end use of the compositions.

The formulations of the present disclosure include at least one cannabis and/or cannabis-derived compound. One particularly suitable cannabis-derived compound for use in the formulations include at least one or more cannabinoid as described more fully below. Particularly suitable cannabinoids include Δ9-tetrahydrocannabinolic acid, Δ9-tetrahydrocannabinol, Δ8-tetrahydrocannabinoilic acid, Δ8-tetrahydrocannabinol, cannabidiolic acid, cannabidiol, cannabigerolic acid, cannabigerol, cannabichromenic acid, cannabichromene, cannabinol, Δ9-tetrahydrocannabivarinic acid, Δ9-tetrahydrocannabivarin, cannabigerivarin, cannabidivarin, cannabichromevarin, 11-hydroxy-Δ9-tetrahydrocannabinol, 11-nor-9-carboxy-Δ9-tetrhydrocannabinol and combinations thereof. Particularly suitable cannabinoids include at least one of cannabidiol (CBD) and Δ9-tetrahydrocannabinol (THC). Other cannabis-derived compounds include cannabis-derived terpenes or terpenoids.

### Cannabis

Cannabis is a genus of flowering plant in the family Cannabaceae. The number of species within the genus is disputed. Three species may be recognized, Cannabis sativa, Cannabis indica and Cannabis ruderalis. C. ruderalis may be included within C. sativa; or all three may be treated as subspecies of a single species, C. sativa. Geoffrey William Guy; Brian Anthony Whittle; Philip Robson (2004); The Medicinal Uses of Cannabis and Cannabinoids. Pharmaceutical Press. pp. 74, et. alia; ISBN 978-0-85369-517-2; “Classification Report”; United States Department of Agriculture; “Indica, Sativa, Ruderalis—Did We Get It All Wrong?”, The Leaf Online; “Species of Cannabis”; GRIN Taxonomy; describe this background information.

The genus is indigenous to central Asia and the Indian subcontinent. A. ElSohly, Mahmoud (2007); Marijuana and the Cannabinoids. Humana Press. p. 8.ISBN 1-58829-456-0, describe this background information. Cannabis has long been used for hemp fiber, for hemp oils, for medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Many additional plants have been selectively bred to produce a maximum of THC (cannabinoids), which is obtained by curing the flowers. Various compounds, including hashish and hash oil, are extracted from the plant. Erowid. 2006. Cannabis Basics, describes this background material.

Within naturally occurring and manmade hybrids, cannabis contains a vast array of compounds. Three compound classes are of interest within the context of the present disclosure although other compounds can be present or added to the compositions to optimize the experience of a given recreational consumer and medical or medicinal patient or patient population. Those classes include cannabinoids, terpenes and flavonoids.

There are many ways of growing cannabis, some of which are natural, and some are carefully designed by Humans, and they will not be recited here. However, one of ordinary skill in the art of cannabis production will typically place a cannabis seed or cutting into a growth media such as soil, manufactured soil designed for cannabis growth or one of many hydroponic growth medias. The cannabis seed or cutting is then provided with water, light and, optionally, a nutrient supplement. At times, the atmosphere and temperature are manipulated to aid in the growth process. Typically, the humidity, air to carbon dioxide gas ratio and elevated temperature, either by use of a heat source or waste heat produced by artificial light, are used. On many occasions ventilation is carefully controlled to maintain the conditions described above within an optimal range to both increase the rate of growth and, optionally, maximize the plant's production of the compounds which comprise the compositions of the disclosure. It is possible to control lighting cycles to optimize various growth parameters of the plant.

Given the number of variables and the complex interaction of the variables, it is possible to develop highly specific formulas for production of cannabis which lead to a variety of desired plant characteristics. At times, these formulae constitute independent inventions. The present disclosure is applicable to use with such inventive means for growing cannabis as well as any of the variety of conventional methods.

Cannabis sativa is an annual herbaceous plant in the Cannabis genus. It is a member of a small, but diverse family of flowering plants of the Cannabaceae family. It has been cultivated throughout recorded history, used as a source of industrial fiber, seed oil, food, recreation, religious and spiritual moods and medicine. Each part of the plant is harvested differently, depending on the purpose of its use.

Cannabis indica, formally known as Cannabis sativa forma indica, is an annual plant in the Cannabaceae family. A putative species of the genus Cannabis.

Cannabis ruderalis is a low-THC species of Cannabis, which is native to Central and Eastern Europe and Russia. It is widely debated as to whether C. ruderalis is a sub-species of Cannabis sativa. Many scholars accept Cannabis ruderalis as its own species due to its unique traits and phenotypes which distinguish it from Cannabis indica and Cannabis sativa.

### Cannabis Derived Compounds

The present disclosure requires cannabis in the form of a living plant to be converted for use in a formulation. The method of conversion typically involves harvesting and, optionally, one of the extraction, fraction, or purification steps described herein. More typically a combination of two or more such steps, more typically yet 2, 3, 4, 5, 6, 7, 8, 9, or 10 individual steps described herein. More typically still a combination of separating the cannabis from the media in which it is grown, drying to reduce the water content, grinding to form a power, extraction and, optionally, a fractionation or purification step is performed.

More typically, the process comprises separation of the cannabis from the media in which is grown followed by 2, 3, 4, or 5 steps as described above are performed, more typically yet, 2, 3, or 4 steps are performed.

Suitably, the cannabis is separated from the media in which it is grown and first dried and then ground. Once in the ground state, it is, optionally, sieved and finally the resins of the plant are extracted. These resins comprise the compositions of the disclosure or additional synthetic or semisynthetic compounds may be added to the resins. Remembering that optional fractionation and purification steps are possible, the compositions of the disclosure may have compounds removed from the resin. At that point, again optionally, synthetic or semisynthetic compounds may be added to the resin to form the compositions of the disclosure.

The specific Embodiments set forth below teach a method of not only identifying useful compositions, but for performing iterative cycles of selection of the formulations of the present disclosure.

Some steps that can optionally be performed to improve the utility of the compositions include addition, removal or control of the absolute concentrations of compounds comprising the compositions, direct breeding of cannabis strains, genetic manipulation by methods known in the field of molecular biology such as gene insertion or deletion, lyophilization and the development of polyploid variants by use of compounds such as colicine.

### Cannabinoids

A cannabinoid is one of a class of diverse chemical compounds that acts on cannabinoid receptors such as CB1 and CB2 in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially as set forth above). The most notable cannabinoid of the phytocannabinoids is tetrahydrocannabinol (THC), the primary psychoactive compound in cannabis. Cannabidiol (CBD) is another cannabinoid that is a major constituent of the plant. There are at least 113 different cannabinoids isolated from cannabis, exhibiting varied effects.

Synthetic cannabinoids and semisynthetic cannabinoids encompass a variety of distinct chemical classes: the classical cannabinoids structurally related to THC, the non-classical cannabinoids (cannabimimetics) including the aminoalkylindoles, 1,5-diarylpyrazoles, quinolines, and arylsulfonamides as well as eicosanoids related to endocannabinoids.

Tetrahydrocannabinol (THC) refers to a psychotropic cannabinoid and is the principal psychoactive constituent of cannabis. Its chemical name is (−)-trans-Δ⁹-tetrahydrocannabinol and the term “THC” is used to refer to isomers as well.

THC in cannabis is assumed to be involved in self-defense, perhaps against herbivores. THC also possesses high UV-B (280-315 nm) absorption properties, which, it has been speculated, could protect the plant from harmful UV radiation exposure.

Cannabidiol (CBD) is one of the active cannabinoids identified in cannabis. It is a major phytocannabinoid, by some accounts making up to 40% of the plant's extract. CBD does not appear to have any intoxicating effects such as those caused by THC in marijuana, but may have effects on anxiety, depression and have an anti-psychotic effect, and have effects on other comorbidities. In some instances, the comorbidities are related to disorders described herein such as pain and post-traumatic stress disorders commonly referred to as “PTSD.”

Cannabinol (CBN) is thought to be a non-psychoactive cannabinoid found only in trace amounts in cannabis and can be produced via oxidative degradation of THCA and THC. Pharmacologically relevant quantities are formed as a metabolite of tetrahydrocannabinol (THC). CBN acts as a partial agonist at the CB1 receptors, but has a higher affinity to CB2 receptors, however; with lower affinities in comparison to THC. Degraded or oxidized cannabis products, such as low-quality baled cannabis and traditionally produced hashish, are high in CBN, but modern production processes have been alleged to minimize the formation of CBN. Cannabinol has been shown to have analgesic properties. Unlike other cannabinoids, CBN does not stem from cannabigerol (CBG).

Cannabigerol (CBG) is thought to be a non-intoxicating cannabinoid found in the Cannabis genus of plants. CBG is the non-acidic form of cannabigerolic acid (CBGA), the parent molecule (“mother cannabinoid”) from which many other cannabinoids are obtained.

CBG has been found to act as a high affinity α2-adrenergic receptor agonist, moderate affinity 5-HT1A receptor antagonist, and low affinity CB1 receptor antagonist. It also binds to the CB2 receptor as an antagonist. Moreover, CBG was without effect up to 80 mg/kg in the mouse tetrad test of cannabimimetic activity (locomotor suppression, catalepsy, hypothermia and analgesia).

Cannabigerolic Acid (CBGA or CBG-A) is the alleged primordial phyto-cannabinoid. It is the alleged compound in cannabis from which all the plant's other naturally occurring cannabinoids are formed; without CBGA, the cannabis plant cannot produce its most useful compounds. It remains one of the most under-studied cannabinoids, with most of current research focusing on the purported healing properties of THC and CBD.

### Terpenes and Terpenoids

Terpenes are a large and diverse class of organic compounds, produced by a variety of plants, particularly conifers, and by some insects such as termites or swallowtail butterflies, which emit terpenes from their osmeteria. They often have a strong odor and may protect the plants that produce them by deterring herbivores and by attracting predators and parasites of herbivores. The difference between terpenes and terpenoids is that terpenes are hydrocarbons, whereas terpenoids contain additional functional groups.

They are the major components of resin, and of turpentine produced from resin. The name “terpene” is derived from the word “turpentine”. In addition to their roles as end-products in many organisms, terpenes are major biosynthetic building blocks within nearly every living creature. Steroids, for example, are derivatives of the triterpene squalene.

When terpenes are modified chemically, such as by oxidation or rearrangement of the carbon skeleton, the resulting compounds are generally referred to as terpenoids. Some authors will use the term terpene to include all terpenoids. Terpenoids are also known as isoprenoids.

Terpenes and terpenoids are the primary constituents of the essential oils of many types of plants and flowers. Essential oils are used widely as fragrances in perfumery, and in medicine and alternative medicines such as aromatherapy. Synthetic variations and derivatives of natural terpenes and terpenoids also greatly expand the variety of aromas used in perfumery and flavors used in food additives. Vitamin A is a terpenoid.

Higher amounts of terpenes are released by trees in warmer weather, acting as a natural form of cloud seeding. The clouds reflect sunlight, allowing the forest to regulate its temperature. The aroma and flavor of hops comes, in part, from sesquiterpenes (mainly alpha-humulene and beta-caryophyllene), which affect beer quality. Terpenes are also major constituents of Cannabis sativa plants, which contain at least 120 identified compounds.

The terpenoids, sometimes called isoprenoids, are a large and diverse class of naturally occurring organic chemicals like terpenes, derived from five-carbon isoprene units assembled and modified in thousands of ways. Most are multicyclic structures that differ from one another not only in functional groups, but also in their basic carbon skeletons. These lipids can be found in all classes of living things, and are the largest group of natural products. About 60% of known natural products are terpenoids. There are virtually countless manmade synthetic and semisynthetic terpenes.

Plant terpenoids are used extensively for their aromatic qualities and play a role in traditional herbal remedies. Terpenoids contribute to the scent of eucalyptus, the flavors of cinnamon, cloves, and ginger, the yellow color in sunflowers, and the red color in tomatoes. Well-known terpenoids include citral, menthol, camphor, salvinorin A in the plant Salvia divinorum, the cannabinoids found in cannabis, ginkgolide and bilobalide found in Ginkgo biloba, and the curcuminoids found in turmeric and mustard seed, and combinations thereof.

The steroids and sterols in animals are biologically produced from terpenoid precursors. Sometimes terpenoids are added to proteins, e.g., to enhance their attachment to the cell membrane; this is known as isoprenylation.

Any terpene can be converted to a terpenoid, synthetic terpenoid or semisynthetic terpenoid by an array of known chemical reactions. These conversions have been taught so exhaustively in the art that one of ordinary skill in synthetic organic chemistry or natural products chemistry would have no difficulty choosing the appropriate steps, sequences of steps and purification means necessary to prepare the subject terpenoid, synthetic or semisynthetic terpenoid and no more will be set forth here.

Exemplary terpenoids include Beta-caryophyllene, Borneol, 1,8-cineole, camphene, Humulene, Limonene, Linalool, Myrcene, Nerolidol, Pulegone, and Terpinolene, and the like, and combinations thereof.

The cannabis and/or cannabis-derived compounds can be used in the formulations alone or with additives that can provide solubility, stability, bioavailability, color, taste and viscosity modifiers.

Suitably, the additives are independently or in combination derived from natural sources, the dosage form is a beverage and the modifiers are selected to be stable in the selected dosage form. More suitably, the dosage form is a beverage and the modifiers are obtained from natural materials. More suitably still, the beverage dosage form complete with modifiers is clear, stable at room temperature and capable of being provided in both bulk and unit dose forms. More suitably yet, the modifiers act synergistically in the dosage form to provide desirable production, storage, distribution or end use.

Suitable additives include: non-cannabis derived terpenes and terpenoids, flavonoids, viscosity modifiers, natural emulsifiers, oils, thickening agents, minerals, acids, bases, vitamins, flavors, colorants, and combinations thereof.

Another suitable embodiment of the oral dosage form of the disclosure provides fast onset of biological effects of cannabis or cannabis derived compositions in Human or animal consumers. The preferred embodiments of the disclosure set forth in the paragraph above are also preferred embodiments of fast acting dosage forms.

### Flavonoids

Flavonoids (or bioflavonoids) (from the Latin word flavus meaning yellow, their color in nature) are a class of plant and fungus secondary metabolites, and can be used as one or more additive in the formulations.

Chemically, flavonoids have the general structure of a 15-carbon skeleton, which consists of two phenyl rings (A and B) and heterocyclic ring (C). This carbon structure can be abbreviated C6-C3-C6. According to the IUPAC nomenclature, they can be classified into: flavonoids or bioflavonoids, isoflavonoids, derived from 3-phenylchromen-4-one (3-phenyl-1,4-benzopyrone) structure, and neoflavonoids, derived from 4-phenylcoumarine (4-phenyl-1,2-benzopyrone) structure.

The three flavonoid classes above are all ketone-containing compounds, and as such, are anthoxanthins (flavones and flavonols). This class was the first to be termed bioflavonoids. The terms flavonoid and bioflavonoid have also been more loosely used to describe non-ketone polyhydroxy polyphenol compounds, which are more specifically termed flavanoids. The three cycle or heterocycles in the flavonoid backbone are generally called ring A, B and C. Ring A usually shows a phloroglucinol substitution pattern.

Flavonoids are widely distributed in plants, fulfilling many functions. Flavonoids are the most important plant pigments for flower coloration, producing yellow or red/blue pigmentation in petals designed to attract pollinator animals. In higher plants, flavonoids are involved in UV filtration, symbiotic nitrogen fixation and floral pigmentation. They may also act as chemical messengers, physiological regulators, and cell cycle inhibitors. Flavonoids secreted by the root of their host plant help Rhizobia in the infection stage of their symbiotic relationship with legumes like peas, beans, clover, and soy. Rhizobia living in soil can sense the flavonoids and triggers the secretion of Nod factors, which in turn are recognized by the host plant and can lead to root hair deformation and several cellular responses such as ion fluxes and the formation of a root nodule. In addition, some flavonoids have inhibitory activity against organisms that cause plant diseases, e.g. Fusarium oxysporum.

Isoflavones use the 3-phenylchromen-4-one skeleton (with no hydroxyl group substitution on carbon at position 2). Examples include: Genistein, Daidzein, Glycitein, Isoflavanes, Isoflavandiols, Isoflavenes, Coumestans, and Pterocarpans.

Exemplary flavonoids include Apigenin, beta-sitosterol, cannaflavin A, kaempferol, luteolin, orientin, and quercetin.

### Dosage Forms

Suitable dosages of the formulations for use in the methods of the present disclosure will depend upon many factors including, for example, age and weight of an individual, at least one precise event requiring professional consultation, severity of an event, specific formulation to be used, nature of a formulation, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

In some embodiments, the formulations of the present disclosure can be administered to a subset of individuals in need thereof as a therapeutic composition. As used herein, an “individual in need” refers to an individual at risk for or having a medical need such as those described herein. Additionally, an “individual in need” is also used herein to refer to an individual at risk for or diagnosed by a medical professional as having a condition described herein. As such, in some embodiments, the methods disclosed herein are directed to a subset of the general population such that, in these embodiments, not all the general population may benefit from the methods. Based on the foregoing, because some of the method embodiments of the present disclosure are directed to specific subsets or subclasses of identified individuals (that is, the subset or subclass of individuals “in need” of assistance in addressing one or more specific conditions noted herein), not all individuals will fall within the subset or subclass of individuals as described herein. Generally, the individual in need is a human. The individual in need can also be, for example, an animal such as a companion animal or a research animal such as, for example, a non-human primate, a mouse, a rat, a rabbit, a cow, a pig, and other types of research animals known to those skilled in the art.

A dosage form is that object delivered to a subject human or non-human organism for testing, placebo, recreational, therapeutic or other use. Widely varying types are set forth below. Countless dosage forms exist. One primary reference describing dosage forms is Remington: The Science and Practice of Pharmacy, 21st ed., ISBN-13 978-0781746731. One of ordinary skill in the pharmaceutical and pharmacological arts are aware of a wide variety of dosage forms and more examples and references need not be set forth here.

Suitable amounts of the cannabis and/or cannabis-derived compounds for use in the formulations of the present disclosure will depend upon many factors including, for example, age and weight of an individual, specific cannabis and/or cannabis-derived compound(s) to be used, nature of a formulation, whether the composition is intended for direct administration or is a concentrate, and combinations thereof. Ultimately, a suitable amount can be readily determined by one skilled in the art. For example, one skilled in the art can begin with a low amount that can be increased until reaching the desired result or effect. Alternatively, one skilled in the art can begin with a high dosage that can be decreased until reaching a minimum dosage needed to achieve the desired result or effect.

As with dosage forms, countless methods of delivery exist. For the purposes of the present disclosure, delivery includes the provision and use of a dosage form containing a composition of the disclosure so that about 10%, 20%, 30%, 40%, 50%, 60%, 70%, or 80%, more suitably, about 90%, and more suitably yet, about 95% of the composition of the disclosure reaches a biological system or material of the subject human or non-human such that it is available for biological interaction with said subject.

Dosage forms used in the disclosure include bioactive substances selected independently or in combination from cannabinoids, terpenoids and flavonoids as described above. Such substances are lipophilic and typically have low solubility in hydrophilic biocompatible matrix materials. One method for obtaining desirable dosage forms comprising lipophilic substances and hydrophilic biocompatible matrix substances is to encapsulate or disperse lipophilic substances in the hydrophilic matrix using additives or modifiers which provide an environment for stable oil-in-water emulsions, micelles, liposomes or other complex phase equilibrium modified compositions. Many of these techniques, modifiers and additives are described herein.

An exemplary method of preparing a stable oil-in-water dosage form is to use a nanoemulsion to encapsulate lipophilic bioactive compounds in a carrier oil. The carrier oil is, optionally, food grade, not adversely affect product quality (such as appearance, taste, texture, or stability), protected from chemical degradation during storage and distribution, and increase bioavailability following ingestion. Carrier oils help stabilize emulsions from Ostwald ripening, a destabilization mechanism of nanoemulsions. This problem arises due to the increased solubility of dispersed phase in a hydrophilic.

A non-limiting list of such exemplary carrier oils includes ethanol, isopropanol, dimethyl sulfoxide, acetone, ethyl acetate, pentane, heptane, diethyl ether, water, medium-chain triglycerides (MCT oil), medium-chain fatty acids (e.g., caproic acid, caprylic acid, capric acid, lauric acid), long-chain triglycerides (LCT oil), long-chain fatty acids (e.g., myristic acid, palmitic acid, stearic acid, arachidic acid, linoleic acid), glycerine/glycerol, maisine cc, glycerol monolinoleate, coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, peppermint oil, hemp oil, sesame oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, LabrafacLipophile WL 1349, Labrail M1944, Peceol, Plurol Oliqiue CC 497, Transcutol HP, Tween 80, Gelucire 48/16, and combinations thereof. In a particularly suitable embodiment, the carrier oil is maisine cc.

Carrier oils can have an effect the physicochemical stability of nanoemulsions in the gastrointestinal tract (GI Tract). The rate and extent of lipid digestion is higher for MCT emulsions than for LCT emulsions, which is attributed to differences in the water dispensability of the medium and long chain fatty acids formed during lipolysis. The total bioavailability of active components after digestion can be higher for LCT emulsions than for MCT emulsions.

Long-chain triglycerides (LCT) contain fatty acids of 12-20 carbon atoms, and can form mixed micelles with a hydrophobic core large enough to accommodate active substances such as THC and other cannabinoids, terpenoids and flavonoids. Medium-chain triglycerides (MCT) contain fatty acids of 12-20 carbon atoms and can form mixed micelles with smaller hydrophobic.

Emulsions can be prepared in concentrated form and later diluted several hundred times in sugar/acid solutions prior to consumption to produce finished dosage forms in either carbonated or non-carbonated biocompatible matrix systems. Selection of an emulsifier can affect the shelf-life and physicochemical properties of the emulsion. Emulsions stabilized by surfactants or other types of stabilizing agents phospholipids, amphiphilic proteins, or polysaccharides, have been developed to provide controlled release, improved entrapment efficiency, and protection from degradation.

Other suitable types of modifiers and additives include natural emulsifiers, oils, thickening agents, minerals, acids, bases, vitamins, flavors, colorants and other processing, storage, distribution, transport, and use conditions such as ultrasonication, nitrogen dosing, packaging, and sterilization.

Emulsions can be prepared several ways such as mechanical processes which employs shear force to break large emulsion droplets into smaller ones, high-pressure homogenization (HPH, including microfluidization) and high-amplitude ultrasonic processing, and ultrasound-assisted emulsification.

Small droplet sizes lead to transparent emulsions. Droplet sizes about 100, 90, 80, 70, 60, 50 or 40 nm are desirable. Suitably the droplet sizes for transparent emulsions are in the range of 40 to 60 nm, more suitably they are 45 to 55 nm, more suitably yet, 50 nm.

When used as beverages, the formulations of the present disclosure comprise a liquid biocompatible matrix prepared for human consumption together with one or more cannabis or cannabis derived compounds. Suitably, such beverage liquids comprise water, oil, or alcohol; with or without additives or modifiers or both. Such beverage liquids can be divided into various groups such as plain water, alcohol, non-alcoholic drink, soft drink, fruit juice, vegetable juice, tea, coffee, milk, or other hot, room temperature or cold liquids used in drinks. Beverages can be caffeinated or non-caffeinated and may contain metapolitically accessible calories or not. Beverages typically comprise a biocompatible liquid, typically water, oil, or alcohol. Such beverages may be produced in ready to use form or be produced in a form suitable for preparation in final consumable form at or proximate to the time of ingestion.

Biocompatible liquids are liquids meeting the common meaning of the term biocompatible. Suitably, biocompatible liquids comprise substances such as water, purified water, distilled water, mineral water, plant oils, glycerin, mineral oil, or any of the presently known biocompatible liquids. Typically, biocompatible liquids will comprise about 50 weight or volume percent of the beverage, more typically, 50, 60, 70, 80, 90, 95 or 98% by weight or volume of the beverage. In one particularly suitable embodiment of the disclosure, the biocompatible liquid is about 95 to 98 weight or volume percent of the beverage and is a non-aqueous material such as a natural oil or other material such as plant oil, glycerin or mineral oil.

Nutritional additives and modifiers comprise substances useful to the consumer of the oral dosage form for maintenance of normal body health. Suitable nutritional additives comprise essential nutrients including vitamins, dietary minerals amino acids and fatty acids vitamin A, vitamin B1, vitamin B2, vitamin B3, vitamin B5, vitamin B6, vitamin B7, vitamin B9, vitamin B12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

Optional additives and modifiers comprise one or more of acids, bases, acidity regulators, alcohol, anticaking agents, antifoaming agents, antioxidants, bulking agents, coagulation agents, food coloring, color retention agents, emulsifiers, flavor enhancers, flour treatment agents, gelling agents, glazing agents, humectants, leavening agents, tracer gases, preservatives, stabilizers, sweeteners, tenderizers, and thickeners.

One class of common additive or modifier useful in oral dosage forms is the group of substances referred to as phospholipids. Phospholipids are made up of two fatty acid tails and a phosphate group head. Fatty acids are long chains mostly made up of hydrogen and carbon, while phosphate groups consist of a phosphorus molecule with four oxygen molecules attached. These two components of the phospholipid are connected via a third molecule, glycerol.

Phospholipids can act as emulsifiers, enabling oils to form a colloid with water. Phospholipids are one of the components of lecithin, which is found in egg-yolks, as well as being extracted from soy beans, and is used as a food additive in many products, and can be purchased as a dietary supplement. Lysolecithins are typically used for water-oil emulsions like margarine, due to their higher HLB ratio.

In one embodiment of the disclosure, phospholipids are additives or modifiers of the oral dosage forms. Typically, such dosage forms are liquids capable of being beverages. Beverages of this type commonly use phospholipid additives or modifiers to solubilize one or more hydrophobic components of the cannabis or cannabis derived composition. The methods of solubilization are described herein. In this embodiment, phospholipids are typically derived from natural sources such as naturally occurring oils from a plant such as coconut, safflower and sunflower. These phospholipids can include secondary products obtained therefrom such as lecithin from sunflower oil. In these embodiments, the phospholipid or derivative therefrom is present in about 0.01-10 weight or volume percent. More typically, 0.01, 0.1, 1 or 10 weight or volume percent, more typically yet 0.1 to 1 weight or volume percent.

In the same fashion as phospholipids, triglycerides are a typical additive or modifier of the oral dosage forms. Triglycerides are chemically tri-esters of fatty acids and glycerol. Triglycerides are formed by combining glycerol with three fatty acid molecules. Alcohols have a hydroxyl (—OH) group. Organic acids have a carboxyl (—COOH) group. Alcohols and organic acids join to form esters. The glycerol molecule has three hydroxyl (—OH) groups. Each fatty acid has a carboxyl group (—COOH). In triglycerides, the hydroxyl groups of the glycerol join the carboxyl groups of the fatty acid to form ester bonds:

HOCH₂CH(OH)CH₂OH+RCO₂H+R′CO₂H+R″CO₂H→RCO₂CH₂CH(O₂CR′)CH₂CO₂R″+3H₂O

The three fatty acids (RCO₂H, R′CO₂H, R″CO₂H in the above equation) are usually different, but many kinds of triglycerides are known. The chain lengths of the fatty acids in naturally occurring triglycerides vary, but most contain 16, 18, or 20 carbon atoms. Natural fatty acids found in plants and animals are typically composed of only even numbers of carbon atoms, reflecting the pathway for their biosynthesis from the two-carbon building-block acetyl CoA. Bacteria, however, possess the ability to synthesize odd- and branched-chain fatty acids. As a result, ruminant animal fat contains odd-numbered fatty acids, such as 15, due to the action of bacteria in the rumen. Many fatty acids are unsaturated, some are polyunsaturated (e.g., those derived from linoleic acid).

Most natural fats contain a complex mixture of individual triglycerides. Because of this, they melt over a broad range of temperatures. Cocoa butter is unusual in that it is composed of only a few triglycerides, derived from palmitic, oleic, and stearic acids in the 1-, 2-, and 3-positions of glycerol, respectively.

In embodiments wherein triglycerides are used as additives or modifiers of the oral dosage form, they are present in about 0.01-10 weight or volume percent. More typically, 0.01, 0.1, 1 or 10 weight or volume percent, more typically yet 0.1 to 1 weight or volume percent.

Natural phospholipid derivatives include egg PC (Egg lecithin), egg PG, soy PC, hydrogenated soy PC, and sphingomyelin. Synthetic phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), phosphatidylserine (DOPS), PEG phospholipid (mPEG-phospholipid, polyglycerin-phospholipid, functionalized-phospholipid, and terminal activated-phospholipid).

Phospholipids can form cell, micelle and liposomal membranes as well as other self-organizing multi-molecular structures because the phosphate group head is hydrophilic (water-loving) while the fatty acid tails are hydrophobic (water-hating). They automatically arrange themselves in a certain pattern in water or other polar environment because of these properties, and form membranes. To form membranes, phospholipids line up next to each other with their heads on the outside of the polar medium and their tails on the inside, thus forming an inner and outer surface. A second layer of phospholipids also forms with heads facing the inside of the structure and tails facing away. In this way, a double layer is formed with phosphate group heads on the outside, and fatty acid tails on the inside. This double layer, called a lipid bilayer, forms the main part of the membrane or other similar structure.

Beverages are packaged as individual packages, suitably single use packages, and multiple packages. The packaging can be in air tight containers. Packaging is comprised of paper, plastic, metal, and glass.

Beverage may include bubble containing or producing liquids with dissolved gas or liquids capable of producing gas proximately in time of consumption. In one aspect of the disclosure, the beverages, optionally comprising additives, modifiers or both, are convenient to consumers and are manufactured at modest expense. Beverages with dissolved gas are created by a method comprising addition of carbon dioxide, ozone, oxygen, and nitrogen. For beverages with dissolved gas, dissolved gas is added to the beverage by methods comprising application of pressure, and adding water with the dissolved gas. The dissolved gas is released from the beverage when pressure is reduced as effervescence.

### Disorders Treated by Formulations of the Present Disclosure

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure. The following disorders are reported to be treated by use of cannabis or cannabis derived compositions. There is no representation herein that cannabis or cannabis derived compositions constituted a medically licensed treatment or cure for any of these disorders in any jurisdiction, only that published information exists for the use of cannabis or cannabis derived compositions for the disorders recited below.

These disorders include: ADD/ADHD, Addiction risk—Physical, Alcoholism, ALS, Alzheimer's, Amotivational Syndrome, Appetite Stimulant, Arthritis, Asthma, Atherosclerosis, Atrophie Blanche, Autism, Cancer—breast, Cancer—colorectal, Cancer—gliomal brain, Cancer—leukemia, Cancer—lung, Cancer—melanoma, Cancer—oral, Cancer-pancreatic, Cancer—prostate, Cancer—Skin, Cancer—Testicular, Chronic Cystitis, COPD, Diabetes, Depression, Dermatitis, Dystonia, Endocannabinoid Deficiency, Epilepsy, Familial Mediterranean Fever, Infertility, Fever, Fibromyalgia, Glaucoma, Heart Disease/Cardiovascular, Hepatitis, Herpes, Hiccups, HIV/AIDS, Hormone disorders, Huntington's Disease, Effects of Hysterectomy, Idiopathic Intracranial Hypertension, Meige's Syndrome, Migraine/Headache, multiple sclerosis, Nausea, Neuronal disorders, Neuropathic pain, Disorders treated by Neuroprotectants, Nutritional disorders, Obesity, Osteoporosis, Pain, Parkinson's Disease, Post-Traumatic Stress Disorder, Pregnancy related disorders, Pruritis, Schizophrenia/Mental disorders, Sickle Cell Disease, Sleep modulation, Spasticity, Spinal Cord Injury, Stroke, Tourette's Syndrome, and Wilson's Disease.

Particularly suitable embodiments of disorders treated by cannabis and cannabis derived compositions include: Acne, ADD and ADHD, Addiction, AIDS, ALS, Alzheimer's Disease, Anorexia, Antibiotic Resistance, Anxiety, Atherosclerosis, Arthritis, Asthma, Autism, Bipolar disorder, Cancer, Digestive Issues, Depression, Diabetes, Endocrine Disorders, Epilepsy and Seizures, Fibromyalgia, Glaucoma, Heart Disease, Huntington's Disease, Inflammation, Irritable Bowel Syndrome, Kidney Disease, Liver Disease, Metabolic Syndrome, Migraine, Mood Disorders, Motion Sickness, Multiple Sclerosis (MS), Nausea, Neurodegeneration, Chronic Pain, Obesity, OCD, Osteoporosis/Bone Health, Parkinson's Disease, Prion/Mad Cow disease, PTSD, Rheumatism, Schizophrenia, Sickle Cell Anemia, Skin Conditions, Sleep Disorders, Spinal Cord Injury, Stress, Stroke and TBI.

### Methods of Making the Formulations of the Disclosure

Extraction in natural products chemistry is a separation process comprising the separation of a substance from a matrix of natural materials and includes liquid-liquid extraction, solid phase extraction and what is commonly referred to as super-critical extraction. The distribution of any given compound or composition (hereinafter desired material) between two phases is an equilibrium condition described by partition theory. This is based on exactly how the desired material moves from a first solution, typically water or other material capable of dissolving a desired material with a first solubility of the desired material, into second material, typically an organic or other immiscible layer having a second solubility of the desired material layer. Super-critical (supercritical) extraction involves entirely different phenomenon and will be described below.

In the context of the present disclosure, the desired material may be a formulation of the disclosure, a subset of compounds which are components of the formulation of the disclosure, or a single compound which is a component of the formulation of the disclosure. Suitably, the desired material is a formulation described herein substantially free of impurities other than solvent or other extraction reagents, more suitably it is about 10%, 20%, 30%, 40%, 50%, 60%, 70%, or 80% free of said impurities, more suitably it is about 90% free of said impurities, more suitably yet, the desired material is about 95% free of impurities other than solvents or other extraction reagents.

There exist several types of extraction, including liquid-liquid extraction, solid-phase extraction, solid-phase microextraction, Soxhlet extraction, fizzy extraction and super-critical CO2 (supercritical carbon dioxide) extraction.

Once various fractions of desired material have been obtained by any method such as any of fractionation and purification methods known in the art, any number of the fractions can be recombined. The recombination can be by simple mixing or by various mechanical.

In some further embodiments, the liquid formulations are further dried to form a powder formulation for use in beverages and foods. The above described formulations may be dried using any method as known in the drying arts to evaporate the water phase of the emulsion, and possibly none, some or essentially all of the carrier solvent. For example, in one embodiment, the formulations are spray dried to form the powder formulation. Alternative methods of preparing the dried powder formulation include, but are not limited to, pan coating, air-suspension coating, centrifugal extrusion, vibrational nozzle technique, freeze-drying or using a food dehydrator.

In some embodiments, the powder formulation can be diluted with a bulking agent or a mixture of bulking agents. Suitable bulking agents include, for example, gum arabic, waxy maize starch, dextrin, maltodextrin, polydextrose, inulin, fructooligosaccharide, sucrose, glucose, fructose, galactose, lactose, maltose, trehalose, cellobiose, lactulose, ribose, arabinose, xylose, lyxose, allose, altrose, mannose, gulose, talose, erythritol, threitol, arabitol, xylitol, mannitol, ribitol, galactitol, fucitol, inositol, maltitol, sorbitol, isomalt, lactitol, polyglycitol, iditol, volemitol, maltotriitol, maltotetraitol, maltol, stevia, stevioside, rebaudioside, neotame, sucralose, saccharin, sodium cyclamate, aspartame, acesulfame potassium, chitin, and chitosan.

In some aspects, the bulking material may comprise a sweetener, pH modifier, pH stabilizer, antimicrobial preservative, antioxidant, texture modifier, colorant or combinations thereof.

In one embodiment of the disclosure, a formulation of the disclosure, a subset of compounds which are components of the formulation of the disclosure, or a single compound which is a component of the formulation of the disclosure, is one or more fractions being recombined.

In one embodiment of the disclosure, a formulation of the disclosure, a subset of compounds which are components of the formulation of the disclosure, or a single compound which is a component of the formulation of the disclosure, is one or more fractions being recombined and another is a synthetic or semisynthetic material such as a synthetic or semisynthetic natural product. In this context, synthetic and semisynthetic means a natural product which has been treated as the terpene as described above. Biosynthetic materials are a particularly desirable material of the disclosure and are used to prepare compositions of the disclosure by any of the recombination methods described herein. Suitable materials with which the biosynthetic materials are combined are any cannabis related compositions described herein as well as synthetic, semisynthetic or other biosynthetic feed stocks.

In a particularly suitable embodiment of the disclosure, two or more fractions, a subset of compounds which are components of the formulation of the disclosure, or a single compound which is a component of the formulation described herein are recombined to form a composition of the disclosure.

1. A liquid formulation comprising at least one cannabis or cannabis derived compound and a biocompatible liquid.

2. The liquid formulation as set forth in claim 1, wherein the cannabis derived compound comprises one or more of a cannabinoid, a terpene, and a terpenoid.

3. The liquid formulation as set forth in claim 1, wherein the cannabinoid is selected from the group consisting of Δ9-tetrahydrocannabinolic acid, Δ9-tetrahydrocannabinol, Δ8-tetrahydrocannabinoilic acid, Δ8-tetrahydrocannabinol, cannabidiolic acid, cannabidiol, cannabigerolic acid, cannabigerol, cannabichromenic acid, cannabichromene, cannabinol, Δ9-tetrahydrocannabivarinic acid, Δ9-tetrahydrocannabivarin, cannabigerivarin, cannabidivarin, cannabichromevarin, 11-hydroxy-Δ9-tetrahydrocannabinol, 11-nor-9-carboxy-Δ9-tetrhydrocannabinol and combinations thereof.

4. The liquid formulation as set forth in claim 1, wherein the cannabinoid is at least one of cannabidiol (CBD) and Δ9-tetrahydrocannabinol (THC).

5. The liquid formulation as set forth in claim 1 further comprising at least one terpene or terpenoid.

6. The liquid formulation as set forth in claim 5, wherein the terpene or terpenoid is selected from the group consisting of citral, menthol, camphor, salvinorin A, ginkgolide and bilobalide, a curcuminoid, beta-caryophyllene, borneol, 1,8-cineole, camphene, humulene, limonene, linalool, myrcene, nerolidol, pulegone, terpinolene, and combinations thereof.

7. The liquid formulation as set forth in claim 1 further comprising an additive selected from the group consisting of non-cannabis derived terpenes and terpenoids, flavonoids, viscosity modifiers, natural emulsifiers, oils, thickening agents, minerals, acids, bases, vitamins, flavors, colorants, and combinations thereof.

8. The liquid formulation as set forth in claim 7 comprising a flavonoid selected from the group consisting of genistein, daidzein, glycitein, isoflavanes, isoflavandiols, isoflavenes, coumestans, pterocarpans, apigenin, beta-sitosterol, cannaflavin A, kaempferol, luteolin, orientin, and quercetin.

9. The liquid formulation as set forth in claim 1, wherein the biocompatible liquid is selected from the group consisting of water, purified water, distilled water, mineral water, plant oils, glycerin, mineral oil and combinations thereof.

10. (canceled)

11. (canceled)

12. (canceled)

13. An emulsion comprising at least one cannabis or cannabis derived compound, a carrier oil, and an aqueous solution.

14. The emulsion as set forth in claim 13, wherein the carrier oil is selected from the group consisting of ethanol, isopropanol, dimethyl sulfoxide, acetone, ethyl acetate, pentane, heptane, diethyl ether, water, medium-chain triglycerides (MCT oil), medium-chain fatty acids (e.g., caproic acid, caprylic acid, capric acid, lauric acid), long-chain triglycerides (LCT oil), long-chain fatty acids (e.g., myristic acid, palmitic acid, stearic acid, arachidic acid, linoleic acid), glycerine/glycerol, maisine cc, glycerol monolinoleate, coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, peppermint oil, hemp oil, sesame oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, LabrafacLipophile WL 1349, Labrail M1944, Peceol, Plurol Oliqiue CC 497, Transcutol HP, Tween 80, Gelucire 48/16, and combinations thereof.

15. The emulsion as set forth in claim 13, wherein the emulsion is a nanoemulsion.

16. The emulsion as set forth in claim 15 comprising a droplet size of from about 40 nm to about 60 nm.

17. A method of preparing the liquid formulation as set forth in claim 1, the method comprising: mixing at least one cannabis or cannabis derived compound and a biocompatible liquid.

18. The method as set forth in claim 17, wherein the mixing comprises one or more process selected from the group consisting of homogenization, microfluidization, high-amplitude ultrasonic processing, and ultrasound-assisted emulsification.

19. The method as set forth in claim 17, wherein the cannabis derived compound comprises one or more of a cannabinoid, a terpene, and a terpenoid.

20. The method as set forth in claim 19, wherein the cannabinoid is selected from the group consisting of Δ9-tetrahydrocannabinolic acid, Δ9-tetrahydrocannabinol, Δ8-tetrahydrocannabinoilic acid, Δ8-tetrahydrocannabinol, cannabidiolic acid, cannabidiol, cannabigerolic acid, cannabigerol, cannabichromenic acid, cannabichromene, cannabinol, Δ9-tetrahydrocannabivarinic acid, Δ9-tetrahydrocannabivarin, cannabigerivarin, cannabidivarin, cannabichromevarin, 11-hydroxy-Δ9-tetrahydrocannabinol, 11-nor-9-carboxy-Δ9-tetrhydrocannabinol and combinations thereof.

21. The method as set forth in claim 17 further comprising mixing an additive into the mixture of the at least one cannabis or cannabis derived compound and biocompatible liquid, the additive being selected from the group consisting of non-cannabis derived terpenes and terpenoids, flavonoids, viscosity modifiers, natural emulsifiers, oils, thickening agents, minerals, acids, bases, vitamins, flavors, colorants, and combinations thereof.

22. The method as set forth in claim 17, wherein the biocompatible liquid is selected from the group consisting of water, purified water, distilled water, mineral water, plant oils, glycerin, mineral oil and combinations thereof.

23. (canceled)

24. (canceled)

25. The method as set forth in claim 17 further comprising drying the liquid formulation to prepare a powder formulation.
